(19) United States
(12) Patent Application Publication
(10) Pub. No.: US 2026/0024634 A1
(43) Pub. Date: Jan. 22, 2026

(54) SYSTEM AND/OR METHOD FOR DETERMINING SERVICE CODES FROM ELECTRONIC SIGNALS AND/OR STATES

(71) Applicant: Akasa, Inc., South San Francisco, CA (US)

(72) Inventors: Varun Granapathi, Union City, CA (US); Jiaming Zeng, Union City, CA (US)

(21) Appl. No.: 18/775,833

(22) Filed: Jul. 17, 2024

(51) Int. Cl. G16H 15/00; G16H 10/60

(52) U.S. Cl. CPC G16H 15/00; G16H 10/60

(57) ABSTRACT

Disclosed are a system, method and apparatus to generate service codes based, at least in part, on electronic documents. In an embodiment, tokens may be embedded in an electronic document based, at least in part, on a linguistic analysis of the electronic document. Likelihoods of applicability of service codes to the electronic document may be determined based, at least in part, on the embedding of tokens.

### BACKGROUND

### 1. Field

This disclosure relates to methods and/or techniques for determining service codes based, at least in part, on expressions in electronic documents.

### 2. Information

Modern services, such as clinical medical services, are typically funded through insurance and/or reimbursement plans. In an implementation, specific types of services may be classified and identified by corresponding service codes. Parties that are to make payment to settle fees for a service provided may then make an amount of payment to a service provider based on service code(s) associated with the service. In the particular example of clinical medical service codes, the continued growth in volume and complexity of clinical service codes is increasingly burdening medical service providers seeking payment for services.

Additionally, machine-learning processes to train neural networks may be applied to a number of uses and/or problem solutions including, for example, advertisement targeting, weather prediction, autonomous vehicle operation, photograph tagging, chess playing, image analysis and classification, recognizing speech, just to provide a few examples. One broad category of trained neural networks includes generative neural network models having a particular function to generate content such as text, images or audio content, just to provide a few examples of content that may be generated by a trained neural network.

### BRIEF DESCRIPTION OF DRAWINGS

Claimed subject matter is particularly pointed out and distinctly claimed in the concluding portion of the specification. However, both as to organization and/or method of operation, together with objects, features, and/or advantages thereof, it may best be understood by reference to the following detailed description if read with the accompanying drawings in which:

FIG. 1 is a schematic diagram of a system to generate codes relating to services according to an embodiment;

FIG. 2A is a flow diagram of a process to determine service codes based on electronic documents, according to particular embodiments;

FIG. 2B is a schematic diagram of a generative neural network model for generating logit values for use in computing a confidence score, according to an embodiment;

FIG. 2C is a visual depiction of a natural language prompt and example response, according to an embodiment;

FIGS. 3 and 4 are flow diagrams of processes to determine service codes based on electronic documents, according to particular embodiments;

FIG. 5 is a flow diagram of a process to training one or more aspects of a generative neural network model, according to an embodiment;

FIG. 6A is a schematic diagram of a generative neural network model, according to one embodiment;

FIG. 6B is a schematic diagram of a generative neural network model, according to another embodiment;

FIG. 7 is a schematic block diagram of an example computing system in accordance with an implementation;

FIG. 8 is a schematic diagram of a neural network formed in “layers”, according to an embodiment; and

FIG. 9 is a flow diagram of an aspect of a training operation, according to an embodiment.

Reference is made in the following detailed description to accompanying drawings, which form a part hereof, wherein like numerals may designate like parts throughout that are corresponding and/or analogous. It will be appreciated that the figures have not necessarily been drawn to scale, such as for simplicity and/or clarity of illustration. For example, dimensions of some aspects may be exaggerated relative to others. Furthermore, structural and/or other changes may be made without departing from claimed subject matter. It should also be noted that directions and/or references, for example, such as up, down, top, bottom, and so on, may be used to facilitate discussion of drawings and are not intended to restrict application of claimed subject matter. Therefore, the following detailed description is not to be taken to limit claimed subject matter and/or equivalents. Further, it is to be understood that other embodiments may be utilized. Also, embodiments have been provided of claimed subject matter and it is noted that, as such, those illustrative embodiments are inventive and/or unconventional; however, claimed subject matter is not limited to embodiments provided primarily for illustrative purposes. Thus, while advantages have been described in connection with illustrative embodiments, claimed subject matter is inventive and/or unconventional for additional reasons not expressly mentioned in connection with those embodiments. In addition, references throughout this specification to “claimed subject matter” refer to subject matter intended to be covered by one or more claims, and are not necessarily intended to refer to a complete claim set, to a particular combination of claim sets (e.g., method claims, apparatus claims, etc.), or to a particular claim.

### DETAILED DESCRIPTION

References throughout this specification to one implementation, an implementation, one embodiment, an embodiment, and/or the like means that a particular feature, structure, characteristic, and/or the like described in relation to a particular implementation and/or embodiment is included in at least one implementation and/or embodiment of claimed subject matter. Thus, appearances of such phrases, for example, in various places throughout this specification are not necessarily intended to refer to the same implementation and/or embodiment or to any one particular implementation and/or embodiment. Furthermore, it is to be understood that particular features, structures, characteristics, and/or the like described are capable of being combined in various ways in one or more implementations and/or embodiments and, therefore, are within intended claim scope. In general, of course, as has always been the case for the specification of a patent application, these and other issues have a potential to vary in a particular context of usage. In other words, throughout the patent application, particular context of description and/or usage provides helpful guidance regarding reasonable inferences to be drawn; however, likewise, “in this context” in general without further qualification refers to the context of the present patent application.

To address burdens in associating services to service codes in billing operations, clinical medicine service providers may employ automated clinical coding (ACC) that uses natural language processing (NLP) to automatically generate diagnosis and procedure medical codes from clinical notes. In an example implementation, computer-assisted coding (CAC) software may scan medical documentation in electronic health records (EHR) to identify essential information and suggest codes for a particular treatment and/or service. A human coder or health care provider may review codes produced by CAC for billing operations, for example. In an embodiment, CAC may reduce an administrative burden on service providers, allowing service providers to increasingly focus on delivering care rather than learning the nuances of coding.

As many healthcare facilities have adopted EHRs and clinicians have become more specific in their documentation efforts, coders have had more content to read/process, slowing down a process of associating codes to records. This occurs while there is a growing pressure to expediate claims to insurance companies to receive quick payment. As such, CAC may streamline coding and eliminate bottlenecks while enabling coders to focus more attention on higher-level audits by reviewing service codes that are generated. Notwithstanding improvements in CAC techniques, CAC techniques may be unable to handle increasingly complex medical notes. For example, for a complicated set of medical notes and/or documents, such CAC techniques may not provide a complete and/or exhaustive expression of diagnosis and/or treatment codes associated with a patient interaction with a medical service provider.

According to an embodiment, a method comprises executing one or more neural networks of a generative neural network model to generate content based, at least in part, on a prompt, the prompt comprising one or more electronic clinical documents regarding one or more patient interactions with one or more medical service providers; and electronically mapping the generated electronic content to one or more service codes in an electronic output document.

In one aspect, determining service codes based, at least in part, on electronic content provided by a generative neural network model may enable a more robust/improved mapping of clinical documents to medical service codes for diagnosis and/or treatment. For example, electronic content from a generative neural network model may enable a identification of additional diagnosis and/or treatment codes than may otherwise be possible with other CAC techniques. This may address problems of other CAC techniques that may miss or underrepresent diagnosis and/or treatment codes relating to a patient's interaction with a medical service provider.

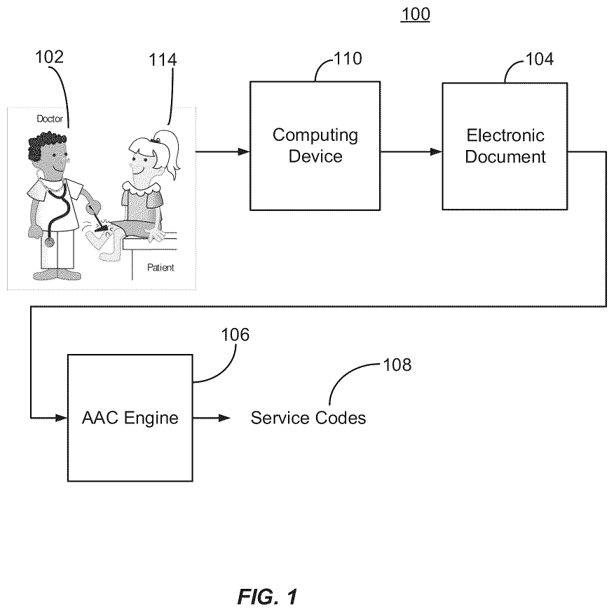

FIG. 1 is a schematic diagram of a system 100 to generate service codes relating to medical services according to an embodiment. While particular features of system 100 may be specifically directed to generation of service codes relating to medical services, it should be understood that aspects may be applied for the generation of service codes descriptive of other, different types of services (e.g., other services for which payment/reimbursement is to be pursued from an insurance company). Here, care provider 102 (e.g., physician, physician assistant, registered nurse, etc.) may be evaluating/tending to patient 114 to, for example, provide a diagnosis and/or treatment. In the course of evaluating/tending to patient 114, care provider 102 may record diagnoses and/or treatments in a patient “chart.” Such a chart may include, for example, notes that are handwritten, typed and/or spoken to be captured by computing device 110. In a particular implementation, computing device 110 may comprise input devices (not shown) such as, for example, a keyboard, microphone and/or scanning device to receive such notes. Notes received at such an input device may then be processed by computer-readable instructions executed by a processor (e.g., to perform speech to text, character recognition, handwriting recognition and/or spell checking) to generate one or more electronic documents 104 expressed as signals and/or states in one or more physical memory devices. In a particular implementation, electronic document 104 may store signals and/or states expressing formatted text to represent notes provided by care provider 102, for example.

AAC engine 106 may comprise one or more computing devices (not shown) to determine service codes 108 based, at least in part, on one or more electronic documents 104. AAC engine 106 may comprise, for example, one or more processors and/or processor executable instructions (not shown) to determine service codes 108 based, at least in part, on one or more electronic documents 104 using natural language processing. In an embodiment, codes 108 may be reviewed, adjusted and/or corrected by care provider 102 and/or other human auditor before submitting service codes 108 to another entity for billing, payment and/or reimbursement.

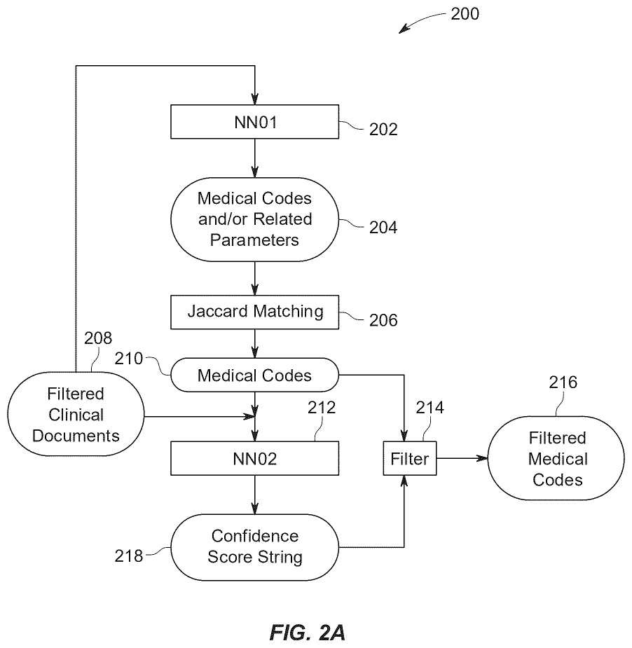

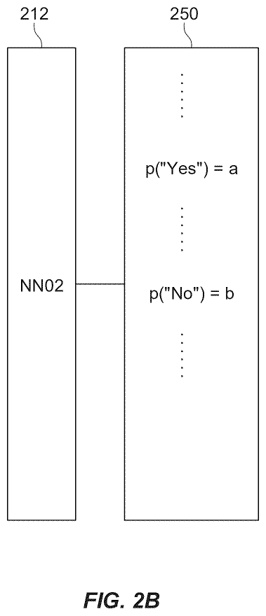

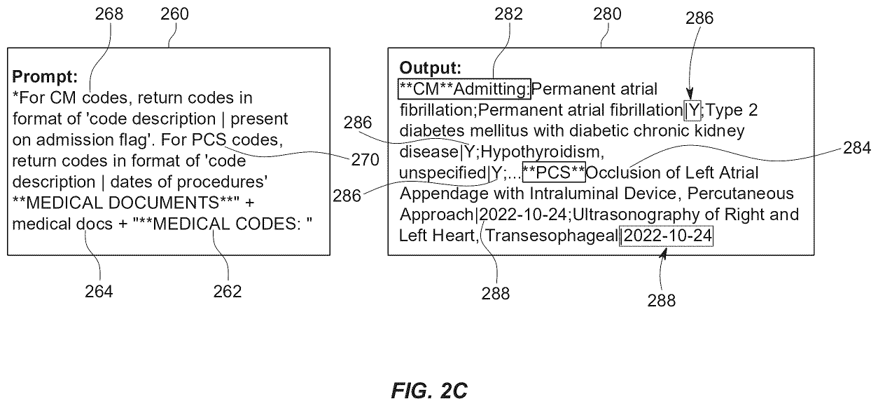

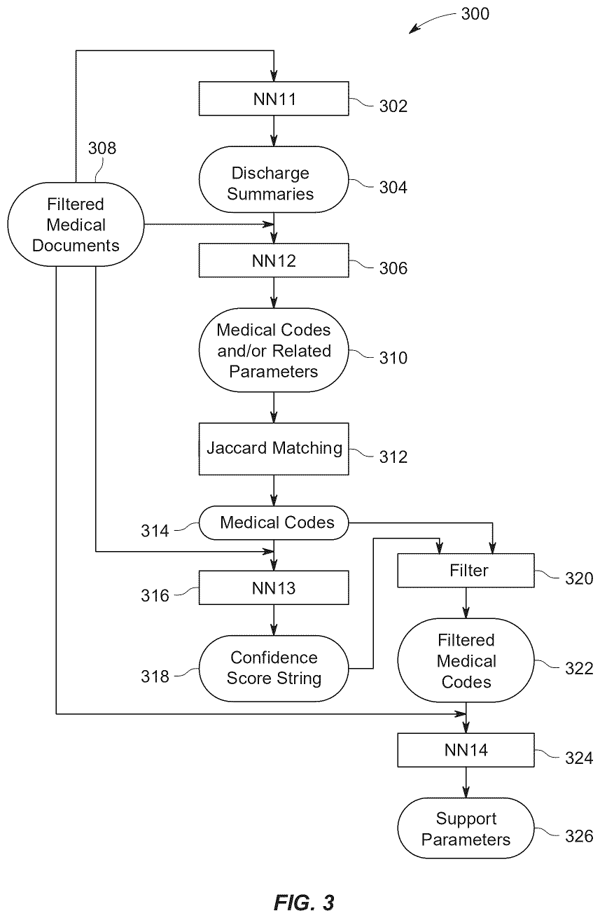

According to an embodiment, ACC engine 106 may be implemented, at least in part, by one or more computing devices executing neural networks. Such neural networks may be implemented, at least in part, according to system 200 shown in FIG. 2 or system 300 shown in FIG. 3. Here, system 200 or 300 may be adapted to generate filtered medical codes 216 or 322 based, at least in part, on filtered clinical documents 208 or 308. Filtered clinical documents 208 or 308 may include, for example, patient records, clinician notes, test results, medical imaging documents, just to provide a few examples. Filtered medical codes 216 or 322 may include, for example, medical diagnosis and/or procedure codes. In one example implementation, such filtered medical codes 216 or 322 may comprise medical codes in a library of medical codes defined by International Classification of Disease (ICD) coding systems, including ICD-10-CM codes for inpatient and outpatient procedures, and ICD-10-PCS codes used for inpatient procedures. For example, such ICD codes may be associated with code titles and/or descriptions such as “single liveborn infant, delivered vaginally” (Z3800), “abnormal findings on neonatal screening for neonatal hearing loss” (P096), “encounter for immunization” (Z23), “immunization not carried out because of caregiver refusal” (Z2882), “encounter for immunization safety counseling” (Z7185), and “neuromyelitis optica [Devic]” (G360). In another example implementation, such filtered medical codes 216 or 322 may be selected from among codes defined in CPT® (Current Procedural Terminology) codes defined by the American Medical Association. For example, such CPT® codes may be associated with code titles and/or descriptions such as, for example, “gastric intubation” (43752, 91105); “interpretation of blood gases and interpretation of data stored in computers, such as ECGs, blood pressure, hematologic data” (99090); “interpretation of cardiac output” (93561-93562); “interpretation of chest X-rays” (71010-71020); “pulse oximetry” (94760-94762); “temporary transcutaneous pacing” (92953); “vascular access procedures” (36000, 36410, 36415, 36591, 36600); and “ventilator management” (94002-94004, 94660, 94662). It should be understood, however, that these are merely examples of how medical codes and related descriptions may be expressed, and that claimed subject matter is not limited in this respect.

According to embodiment, a generative neural network model (e.g., generative neural network model 202, 212, 302, 306, 316 and/or 324) may be configured as generative models according to a large language model (LLM). As referred to herein, a “generative neural network model” means a combination of neural networks having parameters adapted to and/or trained for generation of content such as, for example, image, natural language text, computer code (e.g., pseudocode or source code) and/or audio content, just to provide a few examples. Content generated by such a generative neural network model may be expressed electronically in one or more electrical signals (e.g., in a transmission medium or memory). In particular implementations, generative neural network models referred to herein may be configured from any one of several transformer models including LongT5-3B, MPT-7B, Llama2-7B or Llama2-14B, just to provide a few examples. In an embodiment, generative neural network model 202 may process filtered clinical documents 208 as inputs to produce medical codes and/or related parameters 204. In this example, filtered clinical documents 208 may comprise clinical documents regarding an interaction of a patient with a clinician, such as a doctor's office or other clinic. Medical codes and/or parameters 204 may include, for example, diagnosis codes, treatment codes, procedure codes, relevant billing fields such as discharge status, procedure provider, admitting diagnosis, code titles and/or descriptors, just to provide a few examples. In one implementation, billing fields such as discharge status and procedure provider may be used in other billing operations.

According to an embodiment, generative neural network model 202 may be configured and/or tuned to be non-deterministic to introduce some randomness in generated predictions such as by “increasing a temperature” of the model, for example. As such, generative neural network model 202 may process filtered clinical documents 208 over multiple execution iterations to generate a larger diversity of medical codes and/or related parameters. For example, medical codes and/or related parameters 204 may comprise a union of medical codes and/or related parameters generated from multiple output samples of generative neural network model 202 to process filtered clinical documents 208. Here, multiple output samples of generative neural network model 202 may generate a larger diversity of medical codes and/or related parameters may enable a more complete determination of diagnosis and/or treatment codes associated with an interaction of a patient with a medical service provider.

According to an embodiment, medical codes and/or related parameters 204 may be initially filtered using a Jaccard matching process 206 to provide medical codes 210. Here Jaccard matching process 206 may compare dictionary code titles and/or descriptions of medical codes (e.g., according to ICD and/or CPT®) to code titles and/or descriptions generated by generative neural network model 202 in medical codes and/or related parameters 204 to map individual medical codes and/or related parameters 204 to closest dictionary descriptions. In an example implementation, Jaccard matching process 206 may transform sentence code descriptions in generated medical codes and/or related parameters 204 to first lists of words. Likewise, Jaccard matching process 206 may transform sentence code descriptions in a dictionary code descriptions to second lists of words. Additionally, Jaccard matching process 206 may ensure that words in the first and second lists of words are lower case. For a selected first list (e.g., list of words of a particular generated code description in medical codes and/or related parameters 204), Jaccard matching process 206 may count how many words the selected first list shares with each second list. Based, at least in part on such a count and/or a total number of words in the selected first list and the second list, Jaccard matching process 206 may compute a metric and/or score between zero (lists are most dissimilar) and one (lists are most similar). For pairs of a first list and a second list resulting in scores above a particular threshold value, a medical code corresponding to the second list in the pair may be included as a medical code among medical codes 210 generated by Jaccard matching process 206.

According to an embodiment, generative neural network model 212 may compute a string of confidence scores corresponding to medical codes 210 selected by Jaccard matching process 206. For example, for each medical code 210, generative neural network model 212 may compute logit values a and b based, at least in part, on filtered clinical documents 208 and medical codes 210.

In a particular implementation, a confidence score (CS) for a particular medical code among medical codes 210 may be computed according to expression (1) as follows:

$$CS = \frac{{e}^{a}}{( {e}^{a} + {e}^{b} )} , \qquad ( 1 )$$

where a and b are logit values computed at an output of generative neural network model 212, as shown in FIG. 2B in a particular implementation. For example, an output layer of generative neural network model 212 may provide an output 250 expressing logit value a as a probability or likelihood that an associated predicted code is correct and expressing logit value as a probability or likelihood that the associated predicted code is incorrect. According to an embodiment, CS maps logit values a and b to a probability scale where a value of CS closer to one indicates a high confidence in the particular corresponding medical code among medical codes 210. Conversely, a value of CS closer to zero indicates a low confidence in the particular corresponding medical code among medical codes 210. According to an embodiment, filter 214 may apply confidence scores in confidence score string 218 to corresponding medical codes to 210. For example, medical scores 210 for which a corresponding confidence score 218 is sufficiently high (e.g., exceeds the threshold) may be included among filtered medical codes 216. Conversely, medical scores 210 for which a corresponding confidence score 218 is sufficiently low (e.g., exceeds the threshold) may be excluded from filtered medical codes 216.

FIG. 2C is a diagram illustrating an example natural language prompt 260 that may be processed by system 200 or 300 to provide an output 280 (e.g., an output including filtered medical codes 216). In one particular implementation, natural language prompt may be submitted by a human user via a graphical user interface (GUI). Similarly, output 280 may be presented to such a human user via the GUI. It should be understood, however, that a GUI is merely one example of how a natural language prompt may be received and how a corresponding output may be presented, and that claimed subject matter is not limited in this respect. Prompt 260 may specify particular medical documents 264 (e.g., filtered medical documents 208 or 308) to be used in generating medical codes. In the presently illustrated embodiment, natural language prompt 260 may request one or more clinical modification (CM) codes in a particular format at sentence 268. Sentence 268 also requests a “present on admission” flag to be associated with identified CM codes to indicate whether particular conditions associated with the identified CM codes were present on admission. Natural language prompt 260 also requests procedure coding system (PCS) codes in a particular format at sentence 270. Sentence 270 also requests a “dates of procedures” to be associated with identified PCS codes to indicate dates that associated procedures were performed. Output 280, showing an example response to natural language prompt 260, provides descriptions of identified CM codes 282 annotated with present on admission flags 286 and descriptions of identified PCS codes 284 annotated with dates of procedure indications 288.

In another embodiment, ACC engine 106 may be implemented, at least in part, by one or more computing devices implementing neural networks including generative neural network models in system 300 shown in FIG. 3. Like system 200, system 200 may be adapted to generating filtered medical codes 322 (e.g., as described above for filtered medical codes 216) based, at least in part, on filtered clinical documents 308 (e.g., as described above for filtered clinical documents 208). According to embodiment, generative neural network models 302, 306, 316 and/or 324 may be configured as generative models according to a large language model (LLM), and may be further configured as any one of the several aforementioned transformer models. Generative neural network model 302 may process filtered clinical documents 308 as inputs to produce discharge summaries 304. Such discharge summary for a set of clinical documents associated with a patient encounter with a clinical provider may comprise text to effectively explain to another healthcare professional why the patient was admitted to the clinical provider, what has happened to the patient in the course of the encounter, and other information that may assist in subsequent treatment of the patient. In other words, such a discharge summary may present a concise and comprehensive review of all diagnosis and procedures that has happened to the patient.

Based, at least in part, on discharge summaries 304 and filtered medical documents 308, generative neural network model 306 may generate medical codes and/or related parameters 310 (e.g., as described for medical codes and/or related parameters 204). In one aspect, generating discharge summaries 304 from generative neural network model 306 may inject a degree of entropy that may be beneficial increase a diversity of generated medical codes and/or related parameters 310 at an output of generative neural network model 306. In another aspect, generating discharge summaries 304 from generative neural network model 306 may also inject a level of efficient summarization that decreases an amount of clinical notes to be used for a subsequent operation and otherwise make a process for generation of medical codes more efficient. Like generative neural network model 202, generative neural network model 306 may be configured and/or tuned to be non-deterministic to introduce some randomness in generated predictions such as by “increasing a temperature” of the model, for example. As such, generative neural network model 202 may process filtered clinical documents 308 and/or discharge summaries 304 over multiple execution iterations to generate a larger diversity of medical codes and/or related parameters. For example, medical codes and/or related parameters 310 may comprise a union of medical codes and/or related parameters generated from multiple executions of generative neural network model 306 to process discharge summaries 304 and/or filtered medical documents 308.

According to an embodiment, medical codes and/or related parameters 310 may be initially filtered using a Jaccard matching process 312 (e.g., as described above for Jaccard matching process 206) to provide medical codes 314. Additionally, generative neural network model 316 may compute a confidence score string 318 corresponding to medical codes 314 (e.g., as described for generative neural network 212 computing confidence score string 218). As described above, logit values a and b of an output of generative neural network model 316 may be mapped to a confidence score CS on a probability scale according to expression (1). For each medical code in generated medical codes 314, generative neural network model 212 may compute logit values a and b based, at least in part, on filtered clinical documents 208 and medical codes 210. Filter 320 may apply confidence scores in confidence score string 318 to corresponding medical codes of medical codes 314 to provide filtered medical codes 322 as described above for filter 214.

According to an embodiment, generative neural network model 324 may process filtered medical codes 322 and filtered medical documents 308 to generate support parameters for annotating and/or providing justification for filtered medical codes 322. For example, such support parameters may comprise, for example, an identification of specific documents or portions of documents among filtered medical documents 308 that provide evidence and/or justification for a diagnosis and/or treatment associated with filtered medical condes 322, a textual explanation summarizing such evidence and/or justification, just to provide a few examples.

According to an embodiment, parameters of one or more of generative neural network models 202, 212, 302, 306, 316 and/or 324 may be configured as a natural language processing (NLP) model such as an NLP model capable of generating a text document and selecting materials responsive to a prompt. Such an NLP model may comprise, for example, an NLP model based on word embeddings (e.g., word2vec), large language model (LLM) (e.g., generative pretrained transformer (GPT or Llama-2) model, text-to-text transformer model (e.g., LongT5-3B) or masked language model (e.g., bidirectional encoder representations from transformers (BERT)), just to provide a few examples of an NLP model that may be used.

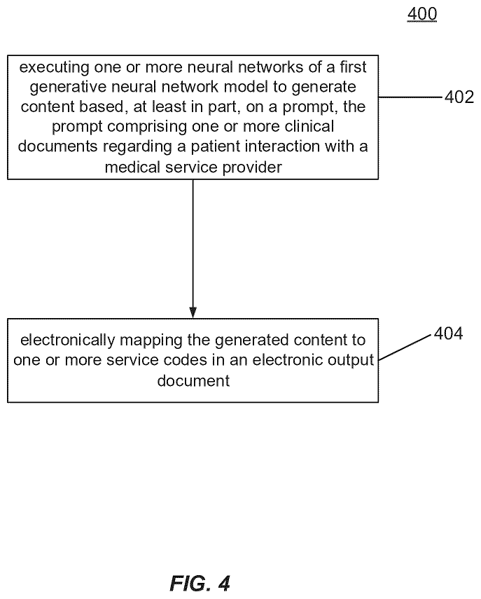

FIG. 4 is a flow diagram of a process 400 for determining service codes from electronic documents, according to an embodiment. Block 402 may comprise executing one or more generative neural networks such as, for example, generative neural network model 202 or 306 to process one or more electronic clinical documents regarding a patient interaction with a medical service provider to generate electronic content (e.g., medical codes and/or related parameters 204 or 310). As pointed out above, such electronic clinical documents may comprise, for example, patient records, clinician notes, test results, medical imaging documents. In one particular implementation, an additional generative neural network model (e.g., generative neural network model 302) may pre-process electronic clinical documents to generate discharge summaries to be inputs to generative neural network model 306.

Block 404 may comprise mapping electronic content generated at block 402 to one or more service codes such as, for example, one or more medical diagnosis and/or treatment codes (e.g., medical codes 210 or 314 and/or filtered medical codes 216 or 322). In one aspect, block 404 may perform a Jaccard matching operation (e.g., Jaccard matching 206 or 312) to map generated medical codes and/or related content to one or more diagnosis and/or treatment codes. In one embodiment, block 404 may perform an additional filtering operation (e.g., by application of filter 214 or 320) based on parameters provided by a generative neural network (e.g., generative neural network 316).

In another embodiment, to complement service codes generated at block 404 an additional generate neural network model (e.g., generative neural network model 324) may be executed to generate electronic content as support parameters (e.g., support parameters 326). As pointed out above, such support parameters may comprise, for example, identification of specific documents or portions of documents among filtered medical documents that provide evidence and/or justification for a diagnosis and/or treatment associated with filtered medical codes, a textual explanation summarizing such evidence and/or justification.

According to an embodiment, process 400 may be initiated by a natural language prompt formulated by an operator (e.g., natural language prompt 260 (FIG. 2C)) at computing device (e.g., via a GUI) comprising, for example, an identity of a patient and records associated with one or more interactions of the patient with one or more medical service providers. Such a prompt may initiate one or more message to a trained generative neural network model to, at least in part, execute blocks 402 and/or 404. Additionally, execution of process 400 may result in the generation of medical codes in a particular output format (e.g., output 280 (FIG. 2C)).

In a particular implementation, parameters of one or more neural networks of a selected NLP model to be implemented in a generative neural network model, such as generative neural network model 202, 212, 302, 306, 316 and/or 324, may be determined in a sequence of training operations. In a first training operation, for example, the selected NLP model may be “pretrained” to recognize and/or interrelate words and phrases relevant to medical diagnoses and/or medical treatments/services. For example, from such a first training operation the selected NLP model may be trained to predict a next/subsequent word based on preceding words.

According to an embodiment, a first training operation to pretrain a selected NLP model may be implemented as a self-supervised training operation and/or an unsupervised training operation. In an unsupervised/self-supervised training operation, a selected NLP model may be furnished with electronic clinical documents including, for example, records of patient encounters available through health care systems. For example, unsupervised/self-supervised training of a selected NLP model may be based on electronic clinical records of patient encounters (e.g., clinician notes, diagnoses, treatments, test results, discharge summaries, medical imaging documents, etc.) obtained from a publicly available corpus of medical documents such as, for example, publicly available records of the Military Health Service (MHS). Such clinical records may be supplemented with other pedagogical materials such as, for example, coding clinic materials, coding guidelines, and/or the like.

Once a selected NLP model is pretrained in a first training operation, the pretrained NLP model may be “fine tuned” to generate desired parameters in an inference stage in one or more subsequent training operations. In an implementation, a single pretrained NLP model may be used to configure multiple generative neural network models in system 200 or system 300. For example, the same pretrained NLP model may be the basis of both generative neural network models 202 and 212 in system 200. Here, the same pretrained NLP model may be separately configured and/or “fine tuned” as generative neural network model 202 and/or generative neural network model 212 in separate subsequent training operations in system 200. Likewise, the same pretrained NLP model may be the basis of generative neural network models 302, 316 and 324 in system 300. Again, the same pretrained NLP model may be separately configured and/or “fine tuned” as generative neural network model 302, 316 and/or 324 in separate subsequent training operations.

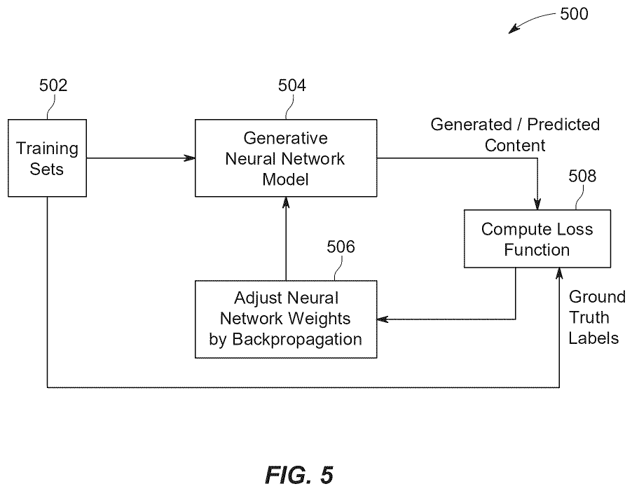

Following an operation to pretrain a generative neural network model, operations to “fine tune” the generative neural network model 202, 212, 302, 306, 316 and/or 324 may be executed according to process 500 shown in FIG. 5. In one example, such a pretrained generative neural network model 504 in a subsequent training operation may be “fine tuned” to generate/predict content (e.g., medical codes and/or related parameters 204 or 510, discharge summaries 304, confidence score strings 218 or 318, and/or support parameters 326) on training epochs/iterations based, at least in part, inputs provided in training sets 502. On training epochs/iterations, such generated/predicted content may be compared with paired ground truth labels in training sets 502 for computation of a loss function at block 508. Neural network weights of generative neural network model 504 may then be adjusted at block 506 using backpropagation, for example, for execution of generative neural network model 504 in a subsequent training epoch/iteration.

In one particular implementation, block 508 may compute a loss function according to one or more particular formulations such as least squares formulation. On any particular training epoch/iteration i, block 508 may compute loss value(s) Cᵢ(yᵢ,ŷᵢ) according to expression (2) as follows:

$${C}_{i} ( {y}_{i} , {\hat{y}}_{i} ) = L ( {y}_{i} , {\hat{y}}_{i} ) , \qquad ( 2 )$$

where:
yᵢ is a ground truth label obtained from training sets 502 for training epoch/iteration i;
ŷᵢ is an output computed by generative neural network model 504 based, at least in part, on input value(s) obtained from training sets 502 for training epoch/iteration i;
L is a suitable loss function.

Block 506 may then adjust/update neural network weights of generative neural network model 504 in a training epoch/iteration i based, at least in part, on a computed value for Cᵢ(yᵢ,ŷᵢ).

In one example, generative neural network model 504 may be fine tuned to generate/predict content including medical codes and/or related parameters (e.g., medical codes and/or related parameters 204 or 310) so as to implement generative neural network model 202 or 306. Here, training sets 502 applied in such a subsequent training operation may comprise sets of clinical documents associated with patient encounters with a health care provider as inputs to generative neural network model 504 on training epochs/iterations, paired with resulting medical codes (e.g., diagnosis and/or treatment codes) and/or related parameters as ground truth labels yᵢ. Such sets of clinical documents paired with resulting medical codes yᵢ in training sets 502 may be obtained from a large public or private corpus of medical documents such as available through MHS. On training epochs/iterations to implement generative neural network model 202 or 306, block 508 may compute a loss value Cᵢ(yᵢ,ŷᵢ) based, at least in part, on medical codes and/or related parameters generated by generative neural network model 504 ŷᵢ paired with medical codes and/or related parameters provided in training sets 502 (provided as ground truth labels yᵢ).

In such a subsequent training operation to configure/fine tune generative neural network model 504 to implement generative neural network model 306, for example, inputs to generative neural network model 504 in training sets 502 may further include discharge summaries for the patient encounters associated with the clinical documents in training sets 502. Here, on training epochs/iterations i to implement generative neural network model 306, block 508 may compute a loss value further based, at least in part, on discharge summaries and medical codes generated by generative neural network model 504 paired with discharge summaries and medical codes provided in training sets 502.

In another example, a pretrained generative neural network model 504 in a subsequent training operation may be “fine tuned” to generate parameters for computing a confidence score (e.g., logit values a and b and/or confidence score string 218 or 318) so as to implement generative neural network model 212 or generative neural network model 316. For example, such a subsequent training operation may comprise applying training sets 502 to include medical codes and associated clinical documents as inputs to generative neural network model 504 on epochs/iterations i, paired with ground truth labels of “Yes” or “No” for each code in training sets 502 as ground truth labels yᵢ on epochs/iterations i. Here, a training set in training sets 502 may comprise a set of clinical documents for a particular patient interaction with one or more correct medical codes as inputs to generative neural network model 504 paired with “Yes” or “No” as a ground truth label yᵢ. Indeed, multiple training sets over multiple training epochs/iterations including clinical documents for multiple patient interactions spanning multiple correct medical codes may be paired with “Yes” as a ground truth label yᵢ. Conversely, additional training sets may include multiple training sets including clinical documents for multiple patient interactions spanning multiple incorrect medical codes as inputs to generative neural network model 504 may be paired with “No” as a ground truth label yᵢ. On training epochs/iterations to implement generative neural network model 212 or 316, block 508 may compute a loss function at block 504 based, at least in part, on logit values a and b generated/predicted by generative neural model 504 as ŷᵢ and logit values a and b provided in training sets 502 as yᵢ.

In another example, a pretrained generative neural network model 504 in a subsequent training operation may be “fine tuned” to generate discharge summaries (e.g., discharge summaries 304) based on filtered medical documents associated with patient encounters with a parameters so as to implement generative neural network model 302. Here, training sets 502 applied in such a subsequent training operation may comprise sets of clinical documents associated with patient encounters with a health care provider as inputs to generative neural network model 504 on epochs/iterations i, paired with discharge summaries based on the patient encounters as ground truth labels yᵢ. Again, such inputs and ground truth labels yᵢ in training sets 502 may be obtained from a large corpus of medical documents such as, for example, publicly available records of the Military Health Service (MHS). On training epochs/iterations to implement generative neural network model 302, block 508 may compute a loss value Cᵢ(yᵢ,ŷᵢ) at block 504 based, at least in part, on discharge summaries generated/predicted by generative neural network model 504 and discharge summaries provided in training sets 502 as ground truth labels yᵢ.

In another example, a pretrained generative neural network model 504 in a subsequent training operation may be “fine tuned” to generate supporting evidence of a medical code so as to implement generative neural network model 324. Such supporting evidence may comprise support parameters 326 to include, for example, an identification of specific documents or portions of documents among filtered medical documents 308 that provide evidence and/or justification for a diagnosis and/or treatment associated with filtered medical codes 322, a textual explanation summarizing such evidence and/or justification for the identified documents or portions thereof. For example, such a subsequent training operation may comprise applying training sets 502 to include medical codes and associated clinical documents as inputs to generative neural network model 504 on iterations/epochs i, paired with text and/or selected portions of the associated clinical documents previously prepared as evidence and/or justification for the medical codes as ground truth labels yᵢ. On training epochs/iterations to implement generative neural network model 324, block 508 may compute a loss value Cᵢ(yᵢ,ŷᵢ) based, at least in part, on text and/or selected portions of the associated clinical documents generated by generative neural network model 504 as ŷᵢ paired with text and/or selected portions of the associated clinical documents previously prepared as evidence and/or justification in training sets 502 as ground truth labels yᵢ.

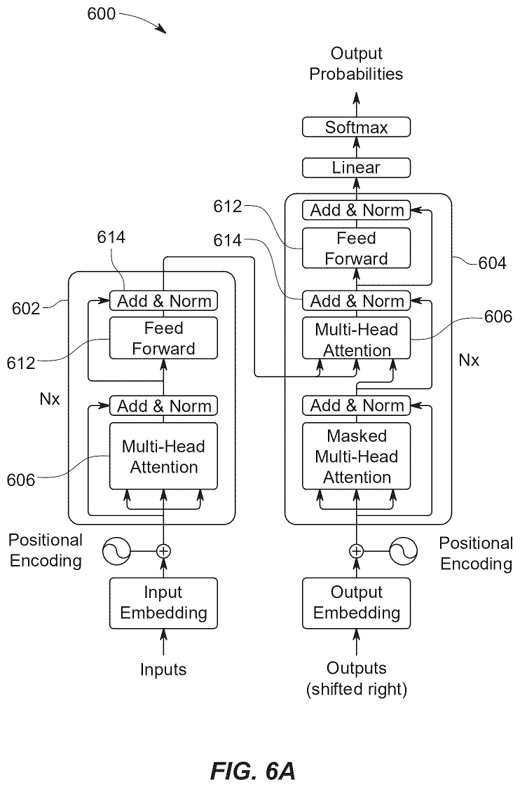

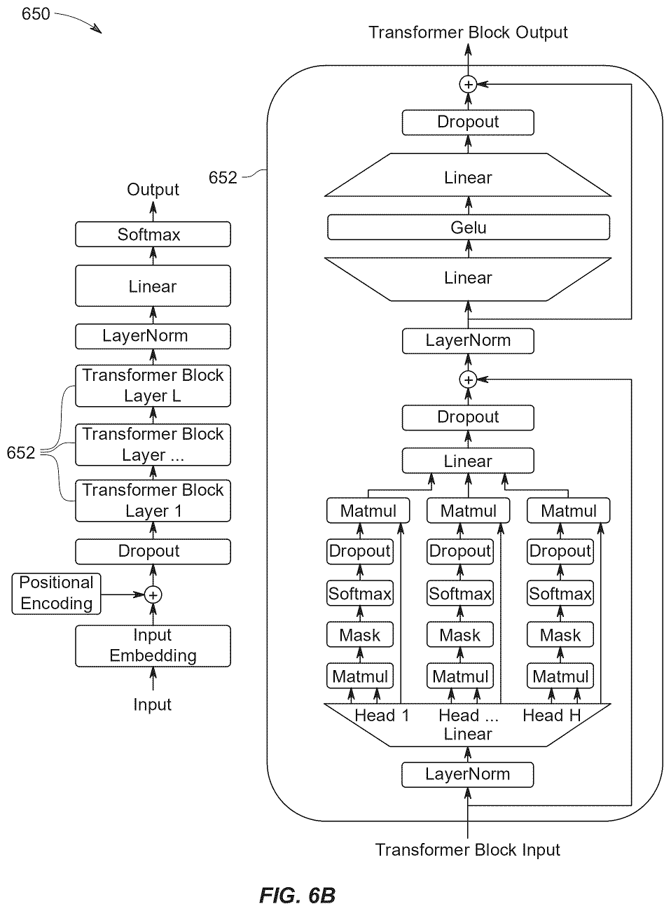

As pointed out above, generative neural network models 202, 212, 302, 306, 316 and 324 may be configured as a natural language processing (NLP) model, such as LLMs powered by versions of models such as LongT5, MPT, and Llama2, just to provide a few examples. FIG. 6A is a schematic diagram of one embodiment of a generative neural network model 600 such as an implementation of generative pretrained model, such as GPT, for example. FIG. 6B is a schematic diagram of another embodiment of generative neural network model 650 as an implementation of a generative pretrained model using a series of transformers 652. In one implementation, inputs generative neural network model 600 and/or 650 may comprise a series of words that are preprocessed (e.g., converted to numbers or other input vectors) and provided in sequence to generate output probabilities of a subsequent word. Such a series of words may be obtained and/or parsed from clinical records of patient encounters from a large corpus of clinical records in a pretraining operation. Once the subsequent word is determined, the subsequent word may be combined with the input so that the next subsequent word may be determined, causing transformers to repeatedly predict a next word in a response to a prompt. In one implementation, an input sequence may be fixed at some value, such as 2048 words, and extra positions at the beginning may be padded with zeros. An output may similarly comprise an array of possible outcomes with associated probabilities, such that the most probable subsequent word may be selected as the next word in the response or output.

Because input vectors in this particular example may indicate only a single word and comprise many more zeros than ones (e.g., GPT has a vocabulary of over 50,000 input words and associated vectors), the input vectors may be embedded or encoded into a smaller multidimensional space at an input embedding element. In generative neural network model 600 in particular, the position of each resulting token in a sequence of inputs may be encoded and provided to a multi-head attention element 606 operable to predict a degree to which an input token is likely to impact an output. Feed-forward blocks 612 may each comprise a multi-layer neural network, operable to learn over time to predict the next word in a sequence. An add & norm block 614 may combine and normalize outputs of multiple previous blocks.

In the context of the present patent application, the term “connection,” the term “component” and/or similar terms are intended to be physical, but are not necessarily always tangible. Whether or not these terms refer to tangible subject matter, thus, may vary in a particular context of usage. As an example, a tangible connection and/or tangible connection path may be made, such as by a tangible, electrical connection, such as an electrically conductive path comprising metal or other conductor, that is able to conduct electrical current between two tangible components. Likewise, a tangible connection path may be at least partially affected and/or controlled, such that, as is typical, a tangible connection path may be open or closed, at times resulting from influence of one or more externally derived signals, such as external currents and/or voltages, such as for an electrical switch. Non-limiting illustrations of an electrical switch include a transistor, a diode, etc. However, a “connection” and/or “component,” in a particular context of usage, likewise, although physical, can also be non-tangible, such as a connection between a client and a server over a network, particularly a wireless network, which generally refers to the ability for the client and server to transmit, receive, and/or exchange communications, as discussed in more detail later.

In a particular context of usage, such as a particular context in which tangible components are being discussed, therefore, the terms “coupled” and “connected” are used in a manner so that the terms are not synonymous. Similar terms may also be used in a manner in which a similar intention is exhibited. Thus, “connected” is used to indicate that two or more tangible components and/or the like, for example, are tangibly in direct physical contact. Thus, using the previous example, two tangible components that are electrically connected are physically connected via a tangible electrical connection, as previously discussed. However, “coupled,” is used to mean that potentially two or more tangible components are tangibly in direct physical contact. Nonetheless, “coupled” is also used to mean that two or more tangible components and/or the like are not necessarily tangibly in direct physical contact, but are able to co-operate, liaise, and/or interact, such as, for example, by being “optically coupled.” Likewise, the term “coupled” is also understood to mean indirectly connected. It is further noted, in the context of the present patent application, since memory, such as a memory component and/or memory states, is intended to be non-transitory, the term physical, at least if used in relation to memory necessarily implies that such memory components and/or memory states, continuing with the example, are tangible.

Unless otherwise indicated, in the context of the present patent application, the term “or” if used to associate a list, such as A, B, or C, is intended to mean A, B, and C, here used in the inclusive sense, as well as A, B, or C, here used in the exclusive sense. With this understanding, “and” is used in the inclusive sense and intended to mean A, B, and C; whereas “and/or” can be used in an abundance of caution to make clear that all of the foregoing meanings are intended, although such usage is not required. In addition, the term “one or more” and/or similar terms is used to describe any feature, structure, characteristic, and/or the like in the singular, “and/or” is also used to describe a plurality and/or some other combination of features, structures, characteristics, and/or the like. Likewise, the term “based on” and/or similar terms are understood as not necessarily intending to convey an exhaustive list of factors, but to allow for existence of additional factors not necessarily expressly described.

The terms “correspond”, “reference”, “associate”, and/or similar terms relate to signals, signal samples and/or states, e.g., components of a signal measurement vector, which may be stored in memory and/or employed with operations to generate results, depending, at least in part, on the above-mentioned, signal samples and/or signal sample states. For example, a signal sample measurement vector may be stored in a memory location and further referenced wherein such a reference may be embodied and/or described as a stored relationship. A stored relationship may be employed by associating (e.g., relating) one or more memory addresses to one or more another memory addresses, for example, and may facilitate an operation, involving, at least in part, a combination of signal samples and/or states stored in memory, such as for processing by a processor and/or similar device, for example. Thus, in a particular context, “associating,” “referencing,” and/or “corresponding” may, for example, refer to an executable process of accessing memory contents of two or more memory locations, e.g., to facilitate execution of one or more operations among signal samples and/or states, wherein one or more results of the one or more operations may likewise be employed for additional processing, such as in other operations, or may be stored in the same or other memory locations, as may, for example, be directed by executable instructions. Furthermore, terms “fetching” and “reading” or “storing” and “writing” are to be understood as interchangeable terms for the respective operations, e.g., a result may be fetched (or read) from a memory location; likewise, a result may be stored in (or written to) a memory location.

It is further noted that the terms “type” and/or “like,” if used, such as with a feature, structure, characteristic, and/or the like, using “optical” or “electrical” as simple examples, means at least partially of and/or relating to the feature, structure, characteristic, and/or the like in such a way that presence of minor variations, even variations that might otherwise not be considered fully consistent with the feature, structure, characteristic, and/or the like, do not in general prevent the feature, structure, characteristic, and/or the like from being of a “type” and/or being “like,” (such as being an “optical-type” or being “optical-like,” for example) if the minor variations are sufficiently minor so that the feature, structure, characteristic, and/or the like would still be considered to be substantially present with such variations also present. Thus, continuing with this example, the terms optical-type and/or optical-like properties are necessarily intended to include optical properties. Likewise, the terms electrical-type and/or electrical-like properties, as another example, are necessarily intended to include electrical properties. It should be noted that the specification of the present patent application merely provides one or more illustrative examples and claimed subject matter is intended to not be limited to one or more illustrative examples; however, again, as has always been the case with respect to the specification of a patent application, particular context of description and/or usage provides helpful guidance regarding reasonable inferences to be drawn.

With advances in technology, it has become more typical to employ distributed computing and/or communication approaches in which portions of a process, such as signal processing of signal samples, for example, may be allocated among various devices, including one or more client devices and/or one or more server devices, via a computing and/or communications network, for example. A network may comprise two or more devices, such as network devices and/or computing devices, and/or may couple devices, such as network devices and/or computing devices, so that signal communications, such as in the form of signal packets and/or signal frames (e.g., comprising one or more signal samples), for example, may be exchanged, such as between a server device and/or a client device, as well as other types of devices, including between wired and/or wireless devices coupled via a wired and/or wireless network, for example.

An example of a distributed computing system comprises the so-called Hadoop distributed computing system, which employs a map-reduce type of architecture. In the context of the present patent application, the terms map-reduce architecture and/or similar terms are intended to refer to a distributed computing system implementation and/or embodiment for processing and/or for generating larger sets of signal samples employing map and/or reduce operations for a parallel, distributed process performed over a network of devices. A map operation and/or similar terms refer to processing of signals (e.g., signal samples) to generate one or more key-value pairs and to distribute the one or more pairs to one or more devices of the system (e.g., network). A reduce operation and/or similar terms refer to processing of signals (e.g., signal samples) via a summary operation (e.g., such as counting the number of students in a queue, yielding name frequencies, etc.). A system may employ such an architecture, such as by marshaling distributed server devices, executing various tasks in parallel, and/or managing communications, such as signal transfers, between various parts of the system (e.g., network), in an embodiment. As mentioned, one non-limiting, but well-known, example comprises the Hadoop distributed computing system. It refers to an open source implementation and/or embodiment of a map-reduce type architecture (available from the Apache Software Foundation, 1901 Munsey Drive, Forrest Hill, MD, 21050-2747), but may include other aspects, such as the Hadoop distributed file system (HDFS) (available from the Apache Software Foundation, 1901 Munsey Drive, Forrest Hill, MD, 21050-2747). In general, therefore, “Hadoop” and/or similar terms (e.g., “Hadoop-type,” etc.) refer to an implementation and/or embodiment of a scheduler for executing larger processing jobs using a map-reduce architecture over a distributed system. Furthermore, in the context of the present patent application, use of the term “Hadoop” is intended to include versions, presently known and/or to be later developed.

In the context of the present patent application, the term network device refers to any device capable of communicating via and/or as part of a network and may comprise a computing device. While network devices may be capable of communicating signals (e.g., signal packets and/or frames), such as via a wired and/or wireless network, they may also be capable of performing operations associated with a computing device, such as arithmetic and/or logic operations, processing and/or storing operations (e.g., storing signal samples), such as in memory as tangible, physical memory states, and/or may, for example, operate as a server device and/or a client device in various embodiments. Network devices capable of operating as a server device, a client device and/or otherwise, may include, as examples, dedicated rack-mounted servers, desktop computers, laptop computers, set top boxes, tablets, netbooks, smart phones, wearable devices, integrated devices combining two or more features of the foregoing devices, and/or the like, or any combination thereof. As mentioned, signal packets and/or frames, for example, may be exchanged, such as between a server device and/or a client device, as well as other types of devices, including between wired and/or wireless devices coupled via a wired and/or wireless network, for example, or any combination thereof. It is noted that the terms, server, server device, server computing device, server computing platform and/or similar terms are used interchangeably. Similarly, the terms client, client device, client computing device, client computing platform and/or similar terms are also used interchangeably. While in some instances, for ease of description, these terms may be used in the singular, such as by referring to a “client device” or a “server device,” the description is intended to encompass one or more client devices and/or one or more server devices, as appropriate. Along similar lines, references to a “database” are understood to mean, one or more databases and/or portions thereof, as appropriate.

It should be understood that for ease of description, a network device (also referred to as a networking device) may be embodied and/or described in terms of a computing device and vice-versa. However, it should further be understood that this description should in no way be construed so that claimed subject matter is limited to one embodiment, such as only a computing device and/or only a network device, but, instead, may be embodied as a variety of devices or combinations thereof, including, for example, one or more illustrative examples.

A network may also include now known, and/or to be later developed arrangements, derivatives, and/or improvements, including, for example, past, present and/or future mass storage, such as network attached storage (NAS), a storage area network (SAN), and/or other forms of device readable media, for example. A network may include a portion of the Internet, one or more local area networks (LANs), one or more wide area networks (WANs), wire-line type connections, wireless type connections, other connections, or any combination thereof. Thus, a network may be worldwide in scope and/or extent. Likewise, sub-networks, such as may employ differing architectures and/or may be substantially compliant and/or substantially compatible with differing protocols, such as network computing and/or communications protocols (e.g., network protocols), may interoperate within a larger network.

The Internet refers to a decentralized global network of interoperable networks that comply with the Internet Protocol (IP). It is noted that there are several versions of the Internet Protocol. The term Internet Protocol, IP, and/or similar terms are intended to refer to any version, now known and/or to be later developed. The Internet includes local area networks (LANs), wide area networks (WANs), wireless networks, and/or long haul public networks that, for example, may allow signal packets and/or frames to be communicated between LANs. The term World Wide Web (WWW or Web) and/or similar terms may also be used, although it refers to a part of the Internet that complies with the Hypertext Transfer Protocol (HTTP). For example, network devices may engage in an HTTP session through an exchange of appropriately substantially compatible and/or substantially compliant signal packets and/or frames. It is noted that there are several versions of the Hypertext Transfer Protocol. The term Hypertext Transfer Protocol, HTTP, and/or similar terms are intended to refer to any version, now known and/or to be later developed. It is likewise noted that in various places in this document substitution of the term Internet with the term World Wide Web (“Web”) may be made without a significant departure in meaning and may, therefore, also be understood in that manner if the statement would remain correct with such a substitution.

The term electronic file and/or the term electronic document are used throughout this document to refer to a set of stored memory states and/or a set of physical signals associated in a manner so as to thereby at least logically form a file (e.g., electronic) and/or an electronic document. That is, it is not meant to implicitly reference a particular syntax, format and/or approach used, for example, with respect to a set of associated memory states and/or a set of associated physical signals. If a particular type of file storage format and/or syntax, for example, is intended, it is referenced expressly. It is further noted an association of memory states, for example, may be in a logical sense and not necessarily in a tangible, physical sense. Thus, although signal and/or state components of a file and/or an electronic document, for example, are to be associated logically, storage thereof, for example, may reside in one or more different places in a tangible, physical memory, in an embodiment.

A Hyper Text Markup Language (“HTML”), for example, may be utilized to specify digital content and/or to specify a format thereof, such as in the form of an electronic file and/or an electronic document, such as a Web page, Web site, etc., for example. An Extensible Markup Language (“XML”) may also be utilized to specify digital content and/or to specify a format thereof, such as in the form of an electronic file and/or an electronic document, such as a Web page, Web site, etc., in an embodiment. Of course, HTML and/or XML are merely examples of “markup” languages, provided as non-limiting illustrations. Furthermore, HTML and/or XML are intended to refer to any version, now known and/or to be later developed, of these languages. Likewise, claimed subject matter are not intended to be limited to examples provided as illustrations, of course.

In the context of the present patent application, the terms “entry,” “electronic entry,” “document,” “electronic document,” “content,”, “digital content,” “item,” and/or similar terms are meant to refer to signals and/or states in a physical format, such as a digital signal and/or digital state format, e.g., that may be perceived by a user if displayed, played, tactilely generated, etc. and/or otherwise executed by a device, such as a digital device, including, for example, a computing device, but otherwise might not necessarily be readily perceivable by humans (e.g., if in a digital format). Likewise, in the context of the present patent application, digital content provided to a user in a form so that the user is able to readily perceive the underlying content itself (e.g., content presented in a form consumable by a human, such as hearing audio, feeling tactile sensations and/or seeing images, as examples) is referred to, with respect to the user, as “consuming” digital content, “consumption” of digital content, “consumable” digital content and/or similar terms. In another embodiment, an electronic document, electronic content and/or digital content may comprise text, audio and/or image content formatted to be processed by a generative neural network model, or text, audio and/or image content generated by a generative neural network model. For one or more embodiments, an electronic document and/or an electronic file may comprise a Web page of code (e.g., computer instructions) in a markup language executed or to be executed by a computing and/or networking device, for example. In another embodiment, an electronic document and/or electronic file may comprise a portion and/or a region of a Web page. However, claimed subject matter is not intended to be limited in these respects.

Also, for one or more embodiments, an electronic document and/or electronic file may comprise a number of components. As previously indicated, in the context of the present patent application, a component is physical, but is not necessarily tangible. As an example, components with reference to an electronic document and/or electronic file, in one or more embodiments, may comprise text, for example, in the form of physical signals and/or physical states (e.g., capable of being physically displayed). Typically, memory states, for example, comprise tangible components, whereas physical signals are not necessarily tangible, although signals may become (e.g., be made) tangible, such as if appearing on a tangible display, for example, as is not uncommon. Also, for one or more embodiments, components with reference to an electronic document and/or electronic file may comprise a graphical object, such as, for example, an image, such as a digital image, and/or sub-objects, including attributes thereof, which, again, comprise physical signals and/or physical states (e.g., capable of being tangibly displayed). In an embodiment, digital content may comprise, for example, text, images, audio, video, and/or other types of electronic documents and/or electronic files, including portions thereof, for example.

Also, in the context of the present patent application, the term parameters (e.g., one or more parameters) refer to material descriptive of a collection of signal samples, such as one or more electronic documents and/or electronic files, and exist in the form of physical signals and/or physical states, such as memory states. For example, one or more parameters, such as referring to an electronic document and/or an electronic file comprising an image, may include, as examples, time of day at which an image was captured, latitude and longitude of an image capture device, such as a camera, for example, etc. In another example, one or more parameters relevant to digital content, such as digital content comprising a technical article, as an example, may include one or more authors, for example. Claimed subject matter is intended to embrace meaningful, descriptive parameters in any format, so long as the one or more parameters comprise physical signals and/or states, which may include, as parameter examples, collection name (e.g., electronic file and/or electronic document identifier name), technique of creation, purpose of creation, time and date of creation, logical path if stored, coding formats (e.g., type of computer instructions, such as a markup language) and/or standards and/or specifications used so as to be protocol compliant (e.g., meaning substantially compliant and/or substantially compatible) for one or more uses, and so forth.

Signal packet communications and/or signal frame communications, also referred to as signal packet transmissions and/or signal frame transmissions (or merely “signal packets” or “signal frames”), may be communicated between nodes of a network, where a node may comprise one or more network devices and/or one or more computing devices, for example. As an illustrative example, but without limitation, a node may comprise one or more sites employing a local network address, such as in a local network address space. Likewise, a device, such as a network device and/or a computing device, may be associated with that node. It is also noted that in the context of this patent application, the term “transmission” is intended as another term for a type of signal communication that may occur in any one of a variety of situations. Thus, it is not intended to imply a particular directionality of communication and/or a particular initiating end of a communication path for the “transmission” communication. For example, the mere use of the term in and of itself is not intended, in the context of the present patent application, to have particular implications with respect to the one or more signals being communicated, such as, for example, whether the signals are being communicated “to” a particular device, whether the signals are being communicated “from” a particular device, and/or regarding which end of a communication path may be initiating communication, such as, for example, in a “push type” of signal transfer or in a “pull type” of signal transfer. In the context of the present patent application, push and/or pull type signal transfers are distinguished by which end of a communications path initiates signal transfer.

Thus, a signal packet and/or frame may, as an example, be communicated via a communication channel and/or a communication path, such as comprising a portion of the Internet and/or the Web, from a site via an access node coupled to the Internet or vice-versa. Likewise, a signal packet and/or frame may be forwarded via network nodes to a target site coupled to a local network, for example. A signal packet and/or frame communicated via the Internet and/or the Web, for example, may be routed via a path, such as either being “pushed” or “pulled,” comprising one or more gateways, servers, etc. that may, for example, route a signal packet and/or frame, such as, for example, substantially in accordance with a target and/or destination address and availability of a network path of network nodes to the target and/or destination address. Although the Internet and/or the Web comprise a network of interoperable networks, not all of those interoperable networks are necessarily available and/or accessible to the public.

A network and/or sub-network, in an embodiment, may communicate via signal packets and/or signal frames, such as via participating digital devices and may be substantially compliant and/or substantially compatible with, but is not limited to, now known and/or to be developed, versions of any of the following network protocol stacks: ARCNET, AppleTalk, ATM, Bluetooth, DECnet, Ethernet, FDDI, Frame Relay, HIPPI, IEEE 1394, IEEE 802.11, IEEE-488, Internet Protocol Suite, IPX, Myrinet, OSI Protocol Suite, QsNet, RS-232, SPX, System Network Architecture, Token Ring, USB, and/or X.25. A network and/or sub-network may employ, for example, a version, now known and/or later to be developed, of the following: TCP/IP, UDP, DECnet, NetBEUI, IPX, AppleTalk and/or the like. Versions of the Internet Protocol (IP) may include IPv4, IPv6, and/or other later to be developed versions.

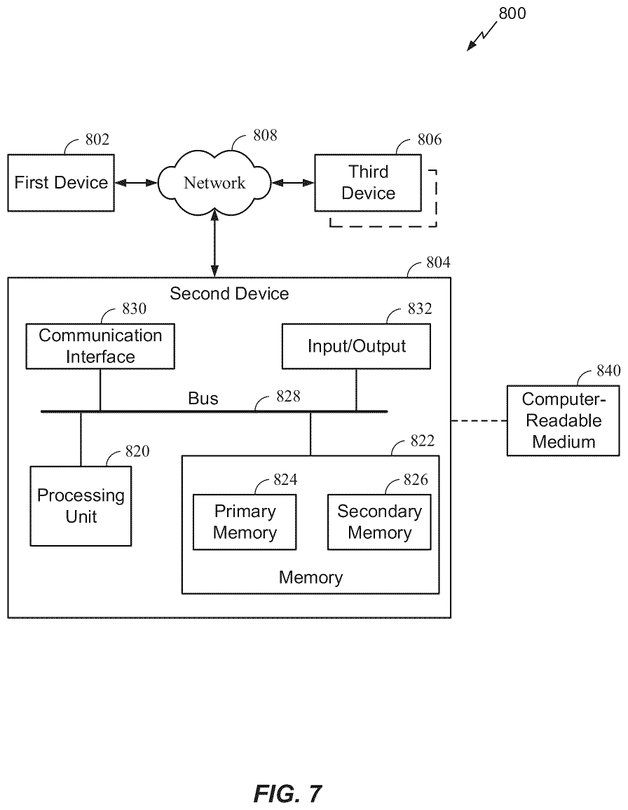

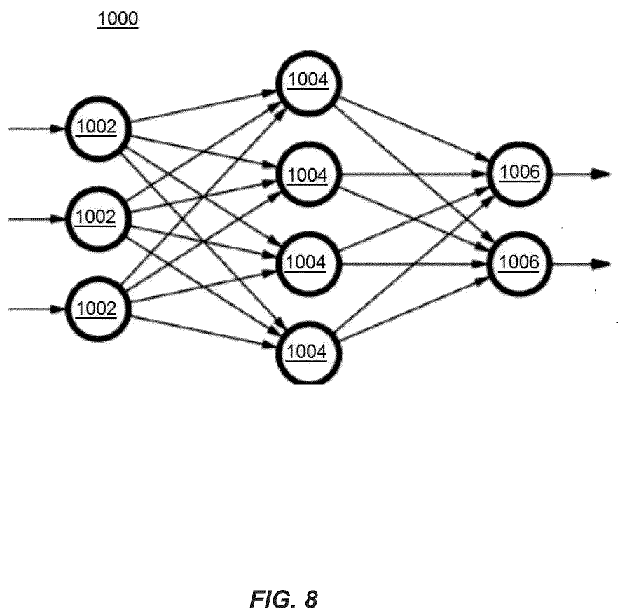

In one example embodiment, as shown in FIG. 7, a system embodiment may comprise a local network (e.g., device 804 and medium 840) and/or another type of network, such as a computing and/or communications network. For purposes of illustration, therefore, FIG. 7 shows an embodiment 800 of a system that may be employed to implement either type or both types of networks. Network 808 may comprise one or more network connections, links, processes, services, applications, and/or resources to facilitate and/or support communications, such as an exchange of communication signals, for example, between a computing device, such as 802, and another computing device, such as 806, which may, for example, comprise one or more client computing devices and/or one or more server computing device. By way of example, but not limitation, network 808 may comprise wireless and/or wired communication links, telephone and/or telecommunications systems, Wi-Fi networks, Wi-MAX networks, the Internet, a local area network (LAN), a wide area network (WAN), or any combinations thereof.

Example devices in FIG. 7 may comprise features, for example, of a client computing device and/or a server computing device, in an embodiment. It is further noted that the term computing device, in general, whether employed as a client and/or as a server, or otherwise, refers at least to a processor and a memory connected by a communication bus. A “processor,” for example, is understood to connote a specific structure such as a central processing unit (CPU) of a computing device which may include a control unit and an execution unit. In an aspect, a processor may comprise a device that fetches, interprets and executes instructions to process input signals to provide output signals. As such, in the context of the present patent application at least, computing device and/or processor are understood to refer to sufficient structure within the meaning of 35 USC § 112 (f) so that it is specifically intended that 35 USC § 112 (f) not be implicated by use of the term “computing device,” “processor” and/or similar terms, however, if it is determined, for some reason not immediately apparent, that the foregoing understanding cannot stand and that 35 USC § 112 (f), therefore, necessarily is implicated by the use of the term “computing device.” “processor” and/or similar terms, then, it is intended, pursuant to that statutory section, that corresponding structure, material and/or acts for performing one or more functions be understood and be interpreted to be described at least in FIGS. 1, 2A, 3, 4, 5, 6A and 6B, and in the text associated with the foregoing figure(s) of the present patent application.

Referring now to FIG. 7, in an embodiment, first and third devices 802 and 806 may be capable of rendering a graphical user interface (GUI) (e.g., including a pointer device) for a network device and/or a computing device, for example, so that a user-operator may engage in system use. Computing device 804 may potentially serve a similar function in this illustration. Likewise, in FIG. 7, computing device 802 (‘first device’ in figure) may interface with computing device 804 (‘second device’ in figure), which may, for example, also comprise features of a client computing device and/or a server computing device, in an embodiment. Processor (e.g., processing device) 820 and memory 822, which may comprise primary memory 824 and secondary memory 826, may communicate by way of a communication bus 815, for example. The term “computing device,” in the context of the present patent application, refers to a system and/or a device, such as a computing apparatus, that includes a capability to process (e.g., perform computations) and/or store digital content, such as electronic files, electronic documents, measurements, text, images, video, audio, etc. in the form of signals and/or states. Thus, a computing device, in the context of the present patent application, may comprise hardware, software, firmware, or any combination thereof (other than software per se). Computing device 804, as depicted in FIG. 7, is merely one example, and claimed subject matter is not limited in scope to this particular example.

For one or more embodiments, a device, such as a computing device and/or networking device, may comprise, for example, any of a wide range of digital electronic devices, including, but not limited to, desktop and/or notebook computers, high-definition televisions, digital versatile disc (DVD) and/or other optical disc players and/or recorders, game consoles, satellite television receivers, cellular telephones, tablet devices, wearable devices, personal digital assistants, mobile audio and/or video playback and/or recording devices, Internet of Things (IOT) type devices, or any combination of the foregoing. Further, unless specifically stated otherwise, a process as described, such as with reference to flow diagrams and/or otherwise, may also be executed and/or affected, in whole or in part, by a computing device and/or a network device. A device, such as a computing device and/or network device, may vary in terms of capabilities and/or features. Claimed subject matter is intended to cover a wide range of potential variations. For example, a device may include a numeric keypad and/or other display of limited functionality, such as a monochrome liquid crystal display (LCD) for displaying text, for example. In contrast, however, as another example, a web-enabled device may include a physical and/or a virtual keyboard, mass storage, one or more accelerometers, one or more gyroscopes, global positioning system (GPS) and/or other location-identifying type capability, and/or a display with a higher degree of functionality, such as a touch-sensitive color 2D or 3D display, for example.

As suggested previously, communications between a computing device and/or a network device and a wireless network may be in accordance with known and/or to be developed network protocols including, for example, global system for mobile communications (GSM), enhanced data rate for GSM evolution (EDGE), 802.11b/g/n/h, etc., and/or worldwide interoperability for microwave access (WiMAX). A computing device and/or a networking device may also have a subscriber identity module (SIM) card, which, for example, may comprise a detachable or embedded smart card that is able to store subscription content of a user, and/or is also able to store a contact list. It is noted, however, that a SIM card may also be electronic, meaning that is may simply be stored in a particular location in memory of the computing and/or networking device. A user may own the computing device and/or network device or may otherwise be a user, such as a primary user, for example. A device may be assigned an address by a wireless network operator, a wired network operator, and/or an Internet Service Provider (ISP). For example, an address may comprise a domestic or international telephone number, an Internet Protocol (IP) address, and/or one or more other identifiers. In other embodiments, a computing and/or communications network may be embodied as a wired network, wireless network, or any combinations thereof.

A computing and/or network device may include and/or may execute a variety of now known and/or to be developed operating systems, derivatives and/or versions thereof, including computer operating systems, such as Windows, iOS, Linux, a mobile operating system, such as iOS, Android, Windows Mobile, and/or the like. A computing device and/or network device may include and/or may execute a variety of possible applications, such as a client software application enabling communication with other devices. A computing and/or network device may also include executable computer instructions to process and/or communicate digital content. A computing and/or network device may also include executable computer instructions to perform a variety of possible tasks, such as browsing, searching, playing various forms of digital content, including locally stored and/or streamed video, and/or games such as, but not limited to, fantasy sports leagues. A computing and/or network device may also process input content as a prompt to one or more generative neural network models to provide output content. A computing and/or network device may also perform linguistic processing such as applying transforms to determine an embedding of tokens and/or apply attention models to determine service codes. The foregoing is provided merely to illustrate that claimed subject matter is intended to include a wide range of possible features and/or capabilities.

In FIG. 7, computing device 802 may provide one or more sources of executable computer instructions in the form physical states and/or signals (e.g., stored in memory states), for example. Computing device 802 may communicate with computing device 804 by way of a network connection, such as via network 808, for example. As previously mentioned, a connection, while physical, may not necessarily be tangible. Although computing device 804 of FIG. 7 shows various tangible, physical components, claimed subject matter is not limited to a computing devices having only these tangible components as other implementations and/or embodiments may include alternative arrangements that may comprise additional tangible components or fewer tangible components, for example, that function differently while achieving similar results. Rather, examples are provided merely as illustrations. It is not intended that claimed subject matter be limited in scope to illustrative examples.

Memory 822 may comprise any non-transitory storage mechanism. Memory 822 may comprise, for example, primary memory 824 and secondary memory 826, additional memory circuits, mechanisms, or combinations thereof may be used. Memory 822 may comprise, for example, random access memory, read only memory, etc., such as in the form of one or more storage devices and/or systems, such as, for example, a disk drive including an optical disc drive, a tape drive, a solid-state memory drive, etc., just to name a few examples.

Memory 822 may be utilized to store a program of executable computer instructions. For example, processor 820 may fetch executable instructions from memory and proceed to interpret and execute the fetched instructions. Memory 822 may also comprise a memory controller for accessing device readable-medium 840 that may carry and/or make accessible digital content, which may include code, and/or instructions, for example, executable by processor 820 and/or some other device, such as a controller, as one example, capable of executing computer instructions, for example. Under direction of processor 820, a non-transitory memory, such as memory cells storing physical states (e.g., memory states), comprising, for example, a program of executable computer instructions, may be executed by processor 820 and able to generate signals to be communicated via a network, for example, as previously described. Generated signals may also be stored in memory, also previously suggested. In a particular implementation, processor 820 may include general processing cores and/or specialized co-processing cores (e.g., signal processors, graphical processing unit (GPU) and/or neural network processing unit (NPU)), for example.

Memory 822 may store electronic files and/or electronic documents, such as relating to one or more users, and may also comprise a computer-readable medium that may carry and/or make accessible content, including code and/or instructions, for example, executable by processor 820 and/or some other device, such as a controller, as one example, capable of executing computer instructions, for example. As previously mentioned, the term electronic file and/or the term electronic document are used throughout this document to refer to a set of stored memory states and/or a set of physical signals associated in a manner so as to thereby form an electronic file and/or an electronic document. That is, it is not meant to implicitly reference a particular syntax, format and/or approach used, for example, with respect to a set of associated memory states and/or a set of associated physical signals. It is further noted an association of memory states, for example, may be in a logical sense and not necessarily in a tangible, physical sense. Thus, although signal and/or state components of an electronic file and/or electronic document, are to be associated logically, storage thereof, for example, may reside in one or more different places in a tangible, physical memory, in an embodiment.

Algorithmic descriptions and/or symbolic representations are examples of techniques used by those of ordinary skill in the signal processing and/or related arts to convey the substance of their work to others skilled in the art. An algorithm is, in the context of the present patent application, and generally, is considered to be a self-consistent sequence of operations and/or similar signal processing leading to a desired result. In the context of the present patent application, operations and/or processing involve physical manipulation of physical quantities. Typically, although not necessarily, such quantities may take the form of electrical and/or magnetic signals and/or states capable of being stored, transferred, combined, compared, processed and/or otherwise manipulated, for example, as electronic signals and/or states making up components of various forms of digital content, such as signal measurements, text, images, video, audio, etc.

It has proven convenient at times, principally for reasons of common usage, to refer to such physical signals and/or physical states as bits, service codes, tokens, computed likelihoods, values, elements, parameters, symbols, characters, terms, numbers, numerals, measurements, content and/or the like. It should be understood, however, that all of these and/or similar terms are to be associated with appropriate physical quantities and are merely convenient labels. Unless specifically stated otherwise, as apparent from the preceding discussion, it is appreciated that throughout this specification discussions utilizing terms such as “processing,” “computing,” “calculating,” “determining”, “establishing”, “obtaining”, “identifying”, “selecting”, “generating”, and/or the like may refer to actions and/or processes of a specific apparatus, such as a special purpose computer and/or a similar special purpose computing and/or network device. In the context of this specification, therefore, a special purpose computer and/or a similar special purpose computing and/or network device is capable of processing, manipulating and/or transforming signals and/or states, typically in the form of physical electronic and/or magnetic quantities, within memories, registers, and/or other storage devices, processing devices, and/or display devices of the special purpose computer and/or similar special purpose computing and/or network device. In the context of this particular patent application, as mentioned, the term “specific apparatus” therefore includes a general purpose computing and/or network device, such as a general purpose computer, once it is programmed to perform particular functions, such as pursuant to program software instructions.

In some circumstances, operation of a memory device, such as a change in state from a binary one to a binary zero or vice-versa, for example, may comprise a transformation, such as a physical transformation. With particular types of memory devices, such a physical transformation may comprise a physical transformation of an article to a different state or thing. For example, but without limitation, for some types of memory devices, a change in state may involve an accumulation and/or storage of charge or a release of stored charge. Likewise, in other memory devices, a change of state may comprise a physical change, such as a transformation in magnetic orientation. Likewise, a physical change may comprise a transformation in molecular structure, such as from crystalline form to amorphous form or vice-versa. In still other memory devices, a change in physical state may involve quantum mechanical phenomena, such as, superposition, entanglement, and/or the like, which may involve quantum bits (qubits), for example. The foregoing is not intended to be an exhaustive list of all examples in which a change in state from a binary one to a binary zero or vice-versa in a memory device may comprise a transformation, such as a physical, but non-transitory, transformation. Rather, the foregoing is intended as illustrative examples.

Referring again to FIG. 7, processor 820 may comprise one or more circuits, such as digital circuits, to perform at least a portion of a computing procedure and/or process. By way of example, but not limitation, processor 820 may comprise one or more processors, such as controllers, microprocessors, microcontrollers, application specific integrated circuits, GPUs, NPUs, digital signal processors, programmable logic devices, field programmable gate arrays, the like, or any combination thereof. In various implementations and/or embodiments, processor 820 may perform signal processing, typically substantially in accordance with fetched executable computer instructions, such as to manipulate signals and/or states, to construct signals and/or states, etc., with signals and/or states generated in such a manner to be communicated and/or stored in memory, for example.

FIG. 7 also illustrates device 804 as including a component 832 operable with input/output devices, for example, so that signals and/or states may be appropriately communicated between devices, such as device 804 and an input device and/or device 804 and an output device. A user may make use of an input device, such as a computer mouse, stylus, track ball, microphone, scanner, keyboard, and/or any other similar device capable of receiving user actions and/or motions as input signals. Likewise, for a device having speech to text capability, a user may speak to a device to generate input signals. A user may make use of an output device, such as a display, a printer, etc., and/or any other device capable of providing signals and/or generating stimuli for a user, such as visual stimuli, audio stimuli and/or other similar stimuli.

In this context, a “neural network” as referred to herein means an architecture of a processing device defined and/or represented by a graph including nodes to represent neurons that process input signals to generate output signals, and edges connecting the nodes to represent input and/or output signal paths between and/or among neurons represented by the graph. In particular implementations, a neural network may comprise a biological neural network, made up of real biological neurons, or an artificial neural network, made up of artificial neurons, for solving artificial intelligence (AI) problems, for example. In an implementation, such an artificial neural network may be implemented by one or more computing devices such as computing devices including a central processing unit (CPU), graphics processing unit (GPU), digital signal processing (DSP) unit and/or neural processing unit (NPU), just to provide a few examples. In a particular implementation, neural network weights and/or numerical coefficients associated with edges to represent input and/or output paths may reflect gains to be applied and/or whether an associated connection between connected nodes is to be excitatory (e.g., weight with a positive value) or inhibitory connections (e.g., weight with negative value). In an example implementation, a neuron may apply a neural network weight to input signals, and sum weighted input signals to generate a linear combination.

According to an embodiment, edges in a neural network connecting nodes may model synapses capable of transmitting signals (e.g., represented by real number values) between neurons. Responsive to receipt of such a signal, a node/neural may perform some computation to generate an output signal (e.g., to be provided to another node in the neural network connected by an edge). Such an output signal may be based, at least in part, on one or more weights and/or numerical coefficients associated with the node and/or edges providing the output signal. For example, such a weight may increase or decrease a strength of an output signal. In a particular implementation, such weights and/or numerical coefficients may be adjusted and/or updated as a machine learning process progresses. In an implementation, transmission of an output signal from a node in a neural network may be inhibited if a strength of the output signal does not exceed a threshold value.

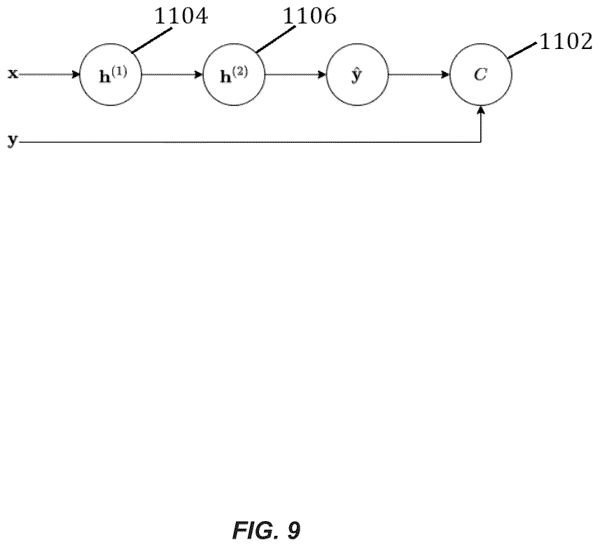

FIG. 9 is a schematic diagram of a neural network 1000 formed in “layers” in which an initial layer is formed by nodes 1002 and a final layer is formed by nodes 1006. All or a portion of features of NN 1000 may be implemented in aspects of systems 200 or 300 such as neural networks making up generative neural network models 202, 212, 302, 306, 316 and/or 324, for example. Neural network (NN) 1000 may include an intermediate layer formed by nodes 1004. Edges shown between nodes 1002 and 1004 illustrate signal flow from an initial layer to an intermediate layer. Likewise, edges shown between nodes 1004 and 1006 illustrate signal flow from an intermediate layer to a final layer. While neural network 1000 shows a single intermediate layer formed by nodes 1004, it should be understood that other implementations of a neural network may include multiple intermediate layers formed between an initial layer and a final layer.

According to an embodiment, a node 1002, 1004 and/or 1006 may process input signals (e.g., received on one or more incoming edges) to provide output signals (e.g., on one or more outgoing edges) according to an activation function. An “activation function” as referred to herein means a set of one or more operations associated with a node of a neural network to map one or more input signals to one or more output signals. In a particular implementation, such an activation function may be defined based, at least in part, on a weight associated with a node of a neural network. Operations of an activation function to map one or more input signals to one or more output signals may comprise, for example, identity, binary step, logistic (e.g., sigmoid and/or soft step), hyperbolic tangent, rectified linear unit, Gaussian error linear unit, Softplus, exponential linear unit, scaled exponential linear unit, leaky rectified linear unit, parametric rectified linear unit, sigmoid linear unit, Swish, Mish, Gaussian and/or growing cosine unit operations. It should be understood, however, that these are merely examples of operations that may be applied to map input signals of a node to output signals in an activation function, and claimed subject matter is not limited in this respect. Additionally, an “activation input value” as referred to herein means a value provided as an input parameter and/or signal to an activation function defined and/or represented by a node in a neural network. Likewise, an “activation output value” as referred to herein means an output value and/or signal provided by an activation function defined and/or represented by a node of a neural network. In a particular implementation, an activation output value may be computed and/or generated according to an activation function based on and/or responsive to one or more activation input values received at a node. In a particular implementation, an activation input value and/or activation output value may be structured, dimensioned and/or formatted as “tensors”. Thus, in this context, an “activation input tensor” or “input tensor” as referred to herein means an expression of one or more activation input values according to a particular structure, dimension and/or format. Likewise in this context, an “activation output tensor” or “output tensor” as referred to herein means an expression of one or more activation output values according to a particular structure, dimension and/or format.

According to an embodiment, neural network 1000 may be characterized as having a particular structure or topology based on, for example, a number of layers, number of nodes in each layers, activation functions implemented at each node, quantization of weights and quantization of input/output activations. Neural network 1000 may be further characterized by weights to be assigned to nodes to affect activation functions at respective nodes. During execution, neural network 1000 may be characterized as having a particular state or “intermediate state” determined based on values/signals computed by nodes (e.g., as activation values to be provided to nodes in a subsequent layer of nodes and/or an output tensor).

In particular implementations, neural networks may enable improved results in a wide range of tasks, including image recognition, speech recognition, content generation, just to provide a couple of example applications. To enable performing such tasks, features of a neural network (e.g., nodes, edges, weights, layers of nodes and edges) may be structured and/or configured to form “filters” that may have a measurable/numerical state such as a value of an output signal. Such a filter may comprise nodes and/or edges arranged in “paths” and are to be responsive to sensor observations provided as input signals. In an implementation, a state and/or output signal of such a filter may indicate and/or infer detection of a presence or absence of a feature in an input signal.

In particular implementations, intelligent computing devices to perform functions supported by neural networks may comprise a wide variety of stationary and/or mobile devices, such as, for example, automobile sensors, biochip transponders, heart monitoring implants, Internet of things (IoT) devices, kitchen appliances, locks or like fastening devices, solar panel arrays, home gateways, smart gauges, robots, financial trading platforms, smart telephones, cellular telephones, security cameras, wearable devices, thermostats, Global Positioning System (GPS) transceivers, personal digital assistants (PDAs), virtual assistants, laptop computers, personal entertainment systems, tablet personal computers (PCs), PCs, personal audio or video devices, personal navigation devices, just to provide a few examples.

According to an embodiment, a neural network may be structured in layers such that a node in a particular neural network layer may receive output signals from one or more nodes in an upstream layer in the neural network, and provide an output signal to one or more nodes in a downstream layer in the neural network. One specific class of layered neural networks may comprise a convolutional neural network (CNN) or space invariant artificial neural networks (SIANN) that enable deep learning. Such CNNs and/or SIANNs may be based, at least in part, on a shared-weight architecture of a convolution kernels that shift over input features and provide translation equivariant responses. Such CNNs and/or SIANNs may be applied to image and/or video recognition, recommender systems, image classification, image segmentation, medical image analysis, natural language processing (e.g., medical records processing), brain-computer interfaces, financial time series, just to provide a few examples.

Another class of layered neural network may comprise a recursive neural network (RNN) that is a class of neural networks in which connections between nodes form a directed cyclic graph along a temporal sequence. Such a temporal sequence may enable modeling of temporal dynamic behavior. In an implementation, an RNN may employ an internal state (e.g., memory) to process variable length sequences of inputs. This may be applied, for example, to tasks such as unsegmented, connected handwriting recognition or speech recognition, just to provide a few examples. In particular implementations, an RNN may emulate temporal behavior using finite impulse response (FIR) or infinite impulse response (IIR) structures. An RNN may include additional structures to control stored states of such FIR and IIR structures to be aged. Structures to control such stored states may include a network or graph that incorporates time delays and/or has feedback loops, such as in long short-term memory networks (LSTMs) and gated recurrent units.

According to an embodiment, output signals of one or more neural networks (e.g., taken individually or in combination) may at least in part, define a “predictor” to generate prediction values associated with some observable and/or measurable phenomenon and/or state. In an implementation, a neural network may be “trained” to provide a predictor that is capable of generating such prediction values based on input values (e.g., measurements and/or observations) optimized according to a loss function. For example, a training process may employ backpropagation techniques. “Backpropagation,” as referred to herein, is to mean a process of fitting parameters of a trained inference model such a model comprising one or more neural networks. In fitting parameters of a neural network, for example, backpropagation is to compute a gradient of a loss function with respect to the weights of the neural network. Based on such a computed gradient of a loss function, weights may be updated so as to minimize and/or reduce such a loss function. In one particular implementation, a gradient descent of a loss function, or variants such as stochastic gradient descent of a loss function, may be used. In training parameters of a neural network, backpropagation may comprise computing a gradient of a loss function with respect to individual weights by the chain rule, computing a gradient one layer at a time, iterating backward from the last layer to avoid redundant calculations of intermediate terms in the chain rule, for example. It should be understood, however, that this is merely an example of how a process of backpropagation may be applied, and claimed subject matter is not limited in this respect. In particular implementations, backpropagation may be used to iteratively update neural network weights to be associated with nodes and/or edges of a neural network based, at least in part on “training sets.” Such training sets may include training measurements and/or observations to be supplied as input values that are paired with “ground truth” observations. Based on a comparison of such ground truth observations and associated prediction values generated based on such input values in a training process, weights may be updated according to a loss function using backpropagation. FIG. 9 is a flow diagram of an aspect of a training operation employing backpropagation to train parameters for a feedforward neural network, according to an embodiment. It should be understood, however, that this is merely an example of a type of neural network that may be trained using backpropagation, and that similar backpropagation techniques may be applied to train parameters of other types of neural networks without deviating from claimed subject matter. Training sets may be provided to such a training operation as pairs of vectors (x,y) where x is an input vector and y is a corresponding ground truth label. Input vector x may be provided as an input tensor to a first hidden layer 1104 to produce an output vector h⁽¹⁾, which is provided as an input to a second hidden layer 1106 to provide an output vector h⁽²⁾. An inference and/or prediction ŷ may be computed based, at least in part, on the output vector h⁽²⁾. A loss value C may be computed at 1102 according to one or more loss functions based, at least in part, on inference and/or prediction ŷ and ground truth label y.

In the particular embodiment of FIG. 9, inference and/or prediction ŷ, and output vectors h⁽¹⁾ and h⁽²⁾ may be modelled as follows:

$${h}^{( 1 )} = {g}^{( 1 )} ( {W}^{( 1 ) T} x + {b}^{( 1 )} )$$

$${h}^{( 2 )} = {g}^{( 2 )} ( {W}^{( 2 ) T} {h}^{( 1 )} + {b}^{( 2 )} )$$

$$\hat{y} ( x ) = {W}^{( 3 ) T} {h}^{( 2 )} + {b}^{( 3 )} ,$$

where:
g⁽ⁱ⁾ is an activation function applied at nodes in hidden layer i;
W⁽ⁱ⁾ is a matrix of weights such that weight

$${W}_{jk}^{( i )}$$

is to be applied at an edge going from node j in layer i−1 to node k in hidden layer i; and
b⁽ⁱ⁾ is a bias matrix applied at hidden layer i.

In a particular implementation in which a feedforward neural network includes three or more hidden layers, computation of ŷ(x) may be generalized as follows:

$$\hat{y} ( x ) = {W}^{( N ) T} {h}^{( N - 1 )} + {b}^{( N )} .$$

Loss value C(y,ŷ) may be computed according to any one of several formulations of a loss function include, for example, a means square error loss or mean absolute error loss, just provide a couple of examples of a loss function. In a particular implementation, a loss function to compute C(y,ŷ) may be differentiable such that

$$\frac{\partial C}{\partial {W}_{jk}^{( i )}}$$

may be determined using the chain rule and may be computed for any weight

$${W}_{jk}^{( i )} .$$

According to an embodiment, values for W⁽ⁱ⁾ may be determined iteratively for training sets (x,y) using a gradient descent technique.

In this context, a “supervised operation” or “unsupervised operation” as referred to herein are to mean a machine-learning operation in which training sets provided as inputs for training iterations are paired with “ground truth” labels. In a training iteration/epoch of such a supervised operation, for example, a loss value may be computed based, at least in part, on an inference computed by a trainable model based on one or more input values a training set and a ground truth label in the training set paired with the one or more input values. For example, a supervised operation may execute a loss function to compute a loss value based, at least in part, on a comparison of a computed inference and ground truth observations/values paired with the computed inference. In this context, a “self-supervised operation” as referred to herein is to mean a machine-learning operation in which input training sets are provided without “ground truth” labels. In a training iteration/epoch of such a self-supervised operation, for example, a loss function may compute a loss value based, at least in part, on an inference computed based on a training set and in the absence of any ground truth label paired with the training set.

One particular embodiment disclosed herein is directed to a computing device comprising: one or more memory devices; and one or more processors to initiate execution of one or more neural networks of a first generative neural network model to generate electronic content based, at least in part, on a prompt, the prompt comprising one or more electronic clinical documents regarding one or more patient interactions with one or more medical service providers; and electronically map the generated electronic content to one or more service codes in an electronic output document. In one particular implementation, the first generative neural network model is trained in multiple training operations comprising a first training operation to train parameters of one or more first neural networks using first training sets, the first training sets comprising electronic clinical records of patient encounters with one or more medical service providers to provide one or more pretrained neural networks; and a second training operation following the first training operation to further train parameters of the one or more pretrained neural networks using second training sets, the second training sets comprising clinical documents and labels associating medical codes and/or medical code descriptions with the clinical documents to provide one or more trained neural networks. In another particular implementation, the first training operation comprises a self-supervised and/or unsupervised training operation. In another particular implementation, the labels further comprise billing information, present on admission status, procedure providers, procedure date and/or discharge status. In yet another particular implementation, the generated electronic content comprises one or more medical codes and/or one or more medical code descriptions, and the one or more processors are further to: electronically mapping the generated electronic content comprises electronically matching the one or more medical codes and/or one or more medical code descriptions to the one or more service codes. In yet another particular implementation, the one or more processors are further to initiate execution of a second generative neural network model to generate parameters to express a confidence in the one or more service codes based, at least in part, on the one or more electronic clinical documents; and filter the one or more service codes based, at least in part, on the parameters to express the confidence, wherein the second generative neural network model is trained in multiple training operations comprising: a first training operation to train parameters of one or more first neural networks using first training sets, the first training sets comprising clinical records of patient encounters with one or more medical service providers to provide one or more pretrained neural networks; and a second training operation following the first training operation to further train the parameters of the one or more pretrained neural networks using second training sets, the second training sets comprising clinical notes and labels associating medical codes and/or medical code descriptions with a confidence metric. In one example, the parameters to express the confidence comprise one or more logit values, and the one or more processors are further to: map the one or more logit values to a value on a probability scale. In another particular implementation, the generated electronic content comprises discharge summaries, and the one or more processors are further to initiate execution of one or more second neural networks to generate medical codes and/or medical descriptions based, at least in part, on the discharge summaries. For example, the first generative neural network model may be trained in multiple training operations comprising: a first training operation to train parameters of one or more first neural networks using first training sets, the first training sets comprising electronic clinical records of patient encounters with one or more medical service providers to provide one or more pretrained neural networks; and a second training operation following the first training operation to further train parameters of the one or more pretrained neural networks using second training sets, the second training sets comprising electronic clinical documents regarding one or more previous patient interactions with one or more medical service providers and labels associating previous discharge summaries with the electronic clinical documents regarding the one or more previous patient interactions. In another particular implementation, the one or more processors are further to initiate execution of one or more neural networks of a second generative neural network model to generate electronic content to express supporting evidence and/or justification for the one or more service codes based, at least in part, on the one or more service codes and at least one of the one or more electronic clinical documents. In one example, the supporting evidence and/or justification may comprise at least an identification of text and/or selected portions of the one or more electronic clinical documents and/or a textual explanation summarizing the evidence and/or justification. In another example, the second generative neural network model may be trained in multiple training operations comprising: a first training operation to train parameters of one or more first neural networks using first training sets, the first training sets comprising electronic clinical records of patient encounters with one or more medical service providers to provide one or more pretrained neural networks; and a second training operation following the first training operation to further train parameters of the one or more pretrained neural networks using second training sets, the second training sets comprising electronic clinical documents regarding one or more previous patient interactions with one or more medical service providers and medical codes as inputs, and labels associating the electronic clinical documents regarding the one or more previous patient interactions and medical codes with previous text summarizing evidence and/or justification for the medical codes.

Another particular embodiment disclosed herein is directed to a computing device comprising: one or more memory devices; and one or more processors to execute a first training operation to train parameters of one or more first neural networks using first training sets, the first training sets comprising electronic clinical records of patient encounters with one or more medical service providers to provide one or more pretrained neural networks; and execute a second training operation following the first training operation to further train parameters of the one or more pretrained neural networks using second training sets, the second training sets comprising clinical documents and labels associating medical codes and/or medical code descriptions with the clinical documents to provide one or more first trained neural networks. In one particular implementation, the first training operation comprises a self-supervised and/or unsupervised training operation. In another particular implementation, the labels further comprise billing information, present on admission status, procedure providers, procedure date and/or discharge status. In yet another particular implementation, the one or more processors are further to execute a third training operation following the first training operation to further train parameters of the one or more pretrained neural networks using second training sets, the second training sets comprising electronic clinical documents regarding one or more previous patient interactions with one or more medical service providers and labels associating previous discharge summaries with the electronic clinical documents regarding the one or more previous patient interactions. In yet another particular implementation, the one or more processors are further to execute a third training operation following the first training operation to further train the parameters of the one or more pretrained neural networks using second training sets, the second training sets comprising electronic clinical documents regarding one or more previous patient interactions with one or more medical service providers and medical codes as inputs, and labels associating the electronic clinical documents regarding the one or more previous patient interactions and medical codes with previous text summarizing evidence and/or justification for the medical codes.

In the preceding description, various aspects of claimed subject matter have been described. For purposes of explanation, specifics, such as amounts, systems and/or configurations, as examples, were set forth. In other instances, well-known features were omitted and/or simplified so as not to obscure claimed subject matter. While certain features have been illustrated and/or described herein, many modifications, substitutions, changes and/or equivalents will now occur to those skilled in the art. It is, therefore, to be understood that the appended claims are intended to cover all modifications and/or changes as fall within claimed subject matter.

What is claimed is:

1. A method comprising:
executing one or more neural networks of a first generative neural network model to generate electronic content based, at least in part, on a prompt, the prompt comprising one or more electronic clinical documents regarding one or more patient interactions with one or more medical service providers; and
electronically mapping the generated electronic content to one or more service codes in an electronic output document.

2. The method of claim 1, wherein the first generative neural network model is trained in multiple training operations comprising:
a first training operation to train parameters of one or more first neural networks using first training sets, the first training sets comprising electronic clinical records of patient encounters with one or more medical service providers to provide one or more pretrained neural networks; and
a second training operation following the first training operation to further train parameters of the one or more pretrained neural networks using second training sets, the second training sets comprising clinical documents and labels associating medical codes and/or medical code descriptions with the clinical documents to provide one or more trained neural networks.

3. The method of claim 2, wherein the first training operation comprises a self-supervised and/or unsupervised training operation.

4. The method of claim 2, wherein the labels further comprise billing information, present on admission status, procedure providers, procedure date and/or discharge status.

5. The method of claim 1, wherein the generated electronic content comprises one or more medical codes and/or one or more medical code descriptions, and the method further comprises:
electronically mapping the generated electronic content comprises electronically matching the one or more medical codes and/or one or more medical code descriptions to the one or more service codes.

6. The method of claim 1, and further comprising:
executing a second generative neural network model to generate parameters to express a confidence in the one or more service codes based, at least in part, on the one or more electronic clinical documents; and
filtering the one or more service codes based, at least in part, on the parameters to express the confidence, wherein the second generative neural network model is trained in multiple training operations comprising:
a first training operation to train parameters of one or more first neural networks using first training sets, the first training sets comprising clinical records of patient encounters with one or more medical service providers to provide one or more pretrained neural networks; and
a second training operation following the first training operation to further train the parameters of the one or more pretrained neural networks using second training sets, the second training sets comprising clinical notes and labels associating medical codes and/or medical code descriptions with a confidence metric.

7. The method of claim 6, wherein the parameters to express the confidence comprise one or more logit values, and the method further comprises:
mapping the one or more logit values to a value on a probability scale.

8. The method of claim 1, wherein the generated electronic content comprises discharge summaries, the method further comprising:
executing one or more second neural networks to generate medical codes and/or medical descriptions based, at least in part, on the discharge summaries.

9. The method of claim 8, wherein the first generative neural network model is trained in multiple training operations comprising:
a first training operation to train parameters of one or more first neural networks using first training sets, the first training sets comprising electronic clinical records of patient encounters with one or more medical service providers to provide one or more pretrained neural networks; and
a second training operation following the first training operation to further train parameters of the one or more pretrained neural networks using second training sets, the second training sets comprising electronic clinical documents regarding one or more previous patient interactions with one or more medical service providers and labels associating previous discharge summaries with the electronic clinical documents regarding the one or more previous patient interactions.

10. The method of claim 1, and further comprising:
executing one or more neural networks of a second generative neural network model to generate electronic content to express supporting evidence and/or justification for the one or more service codes based, at least in part, on the one or more service codes and at least one of the one or more electronic clinical documents.

11. The method of claim 10, wherein the supporting evidence and/or justification comprises at least an identification of text and/or selected portions of the one or more electronic clinical documents and/or a textual explanation summarizing the evidence and/or justification.

12. The method of claim 10, wherein the second generative neural network model is trained in multiple training operations comprising:
a first training operation to train parameters of one or more first neural networks using first training sets, the first training sets comprising electronic clinical records of patient encounters with one or more medical service providers to provide one or more pretrained neural networks; and
a second training operation following the first training operation to further train parameters of the one or more pretrained neural networks using second training sets, the second training sets comprising electronic clinical documents regarding one or more previous patient interactions with one or more medical service providers and medical codes as inputs, and labels associating the electronic clinical documents regarding the one or more previous patient interactions and medical codes with previous text summarizing evidence and/or justification for the medical codes.

13. An article, comprising:
a storage medium comprising computer-readable instructions stored thereon, the instructions to be executable by one or more processors of a computing device to:
initiate execution of one or more neural networks of a first generative neural network model to generate electronic content based, at least in part, on a prompt, the prompt comprising one or more electronic clinical documents regarding one or more patient interactions with one or more medical service providers; and
electronically map the generated electronic content to one or more service codes in an electronic output document.

14. The article of claim 13, wherein the first generative neural network model is trained in multiple training operations comprising:
a first training operation to train parameters of one or more first neural networks using first training sets, the first training sets comprising electronic clinical records of patient encounters with one or more medical service providers to provide one or more pretrained neural networks; and
a second training operation following the first training operation to further train parameters of the one or more pretrained neural networks using second training sets, the second training sets comprising clinical documents and labels associating medical codes and/or medical code descriptions with the clinical documents to provide one or more trained neural networks.

15. The article of claim 13, wherein the instructions are further executable by the one or more processors of the computing device to:
initiate execution of a second generative neural network model to generate parameters to express a confidence in the one or more service codes based, at least in part, on the one or more electronic clinical documents; and
filter the one or more service codes based, at least in part, on the parameters to express the confidence, wherein the second generative neural network model is trained in multiple training operations comprising:
a first training operation to train parameters of one or more first neural networks using first training sets, the first training sets comprising clinical records of patient encounters with one or more medical service providers to provide one or more pretrained neural networks; and
a second training operation following the first training operation to further train the parameters of the one or more pretrained neural networks using second training sets, the second training sets comprising clinical notes and labels associating medical codes and/or medical code descriptions with a confidence metric.

16. A method, executed by one or more processors of a computing device, comprising:
executing a first training operation to train parameters of one or more first neural networks using first training sets, the first training sets comprising electronic clinical records of patient encounters with one or more medical service providers to provide one or more pretrained neural networks; and
executing a second training operation following the first training operation to further train parameters of the one or more pretrained neural networks using second training sets, the second training sets comprising clinical documents and labels associating medical codes and/or medical code descriptions with the clinical documents to provide one or more first trained neural networks.

17. The method of claim 16, wherein the first training operation comprises a self-supervised and/or unsupervised training operation.

18. The method of claim 16, wherein the labels further comprise billing information, present on admission status, procedure providers, procedure date and/or discharge status.

19. The method of claim 16, and further comprising:
executing a third training operation following the first training operation to further train parameters of the one or more pretrained neural networks using second training sets, the second training sets comprising electronic clinical documents regarding one or more previous patient interactions with one or more medical service providers and labels associating previous discharge summaries with the electronic clinical documents regarding the one or more previous patient interactions.

20. The method of claim 16, and further comprising:
executing a third training operation following the first training operation to further train the parameters of the one or more pretrained neural networks using second training sets, the second training sets comprising electronic clinical documents regarding one or more previous patient interactions with one or more medical service providers and medical codes as inputs, and labels associating the electronic clinical documents regarding the one or more previous patient interactions and medical codes with previous text summarizing evidence and/or justification for the medical codes.
